(19) United States
(12) Patent Application Publication
(10) Pub. No.: US 2012/0089052 A1
(43) Pub. Date: Apr. 12, 2012

(54) Massaging device with multiple ultrasonic transducers

(76) Inventors: Yuchen Zhou, San Jose, CA (US); Hieu Tieu, San Jose, CA (US); Chao Uei Wahng, Fremont, CA (US)

(21) Appl. No.: 12/932,316

(22) Filed: Feb. 22, 2011

Related U.S. Application Data
(60) Provisional application No. 61/404,923, filed on Oct. 12, 2010.

(51) Int. Cl. A61N 7/00

(52) U.S. Cl. 601/2

(57) ABSTRACT

The present invention teaches an ultrasonic device for skin care massage, comprising one or more ultrasonic vibration transmission plates, each of which having a backside and a smooth surface for contacting human skin; and at least two different ultrasonic vibration transducers coupled to a backside of each of the ultrasonic vibration plates. Each of the ultrasonic vibration transducers generates an ultrasonic wave with a unique frequency between 20 kHz to 25 MHz. Each of the ultrasonic vibration transducers has a bottom surface paralleling to the smooth surface. A distance between the bottom surface of any of the ultrasonic vibration transducers to the smooth surface is approximately an integer times of the half wavelength of the ultrasonic wave generated by the ultrasonic vibration transducer.

### REFERENCE TO RELATED APPLICATION

The present application claims priority to the provisional Appl. Ser. No. 61/404,923 filed on Oct. 12, 2010, the entire content of which is hereby incorporated by reference.

### FIELD OF THE INVENTION

The invention relates to the technology of ultrasonic implementation in massaging devices. More particularly, the invention relates to an ultrasonic device with a single vibration transmission plate which has multiple ultrasonic modes and can provide vibrations in deferent frequencies.

### BACKGROUND AND PRIOR ARTS

Ultrasound method used for skin care and beautification has been long demonstrated and realized in commercial products and therapeutic devices. The following listed documents are deemed to be the references which are most relevant to the subject matter of the present invention: [1] Y. Mitsu, “Skin beautification cosmetic system using iontophoresis device, ultrasonic facial stimulator, and cosmetic additive”, Pat. No. 7,427,273 B2 (2008); [2] M. Nunomura, et al, “Ultrasound applying skin care device”, Pub. No. US 2006/0149169 (2006); [3] U. Motoyoshi, “ULTRASONIC FACIAL AND BEAUTY APPLIANCE”, Pub. No. JP2007050204 (A) (2007); [4] H. Hisao, “ULTRASONIC FACE MASSAGER”, Pub. No. JP2001314473 (A) (2001); [5] J. Reed, et al, “Ultrasound based cosmetic therapy method and apparatus”, Pub. No. US 2009/0318853 (2009); [6] FaceMate 330 Ultrasonic Face and Skin Massager from Balkowitsch Enterprises (see www.balkowitsch.com); and [7] S. H. Dayan, “Ultrasound-assisted Facial Skin Rejuvenation”, Skin Inc. Magazine (2001).

All existing products share a common feature of single ultrasound transducer exciting a single vibration plate with a roughly same or similar size as the transducer. FIG. 1 shows a schematic of such kind of design according to the prior arts. The vibration plate 1, which is usually metallic, contacts human skin with its outside smooth surface and is driven by an ultrasonic transducer 2 at the inside surface with an ultrasonic frequency. The vibration from the transducer 2 is transmitted to the human skin or skin care products between the outside surface of the plate 1 and the human skin via the driven vibration of the plate 1. The plate 1 and the transducer 2 are generally contained within an enclosed housing structure 3 for handling.

The device according to the prior arts mentioned above only provides vibration with a single frequency. Scientific researches demonstrate that one benefits from ultrasonic treatment of skin for beautification purpose. It is desired that multiple frequencies are used in the ultrasonic device. For example, a higher frequency ultrasonic mode activates the skin care specimen molecules while a lower frequency mode pushes the active ingredients of the specimen deeper into the skin to enhance the beautification effect.

For some of the existing products, such as the device described in FaceMate 330 Ultrasonic Face and Skin Massager from Balkowitsch Enterprises (www.balkowitsch.com), two ultrasound modes are adopted. The devices utilize two separate vibrating plates, with each plate having an ultrasound transducer vibrating at a different frequency. Such design although provides options of two distinctive ultrasound modes, where the higher frequency ultrasound mode acts superficially to activate molecules of beauty products, and the lower frequency mode enables deeper penetration of the product for better beautification result with the lower frequency mode, it has the intrinsic flaw of requiring user to manually shift plates to the target skin area to benefit the functions of different ultrasound modes. Such requirement is not only creating a tedious task for the user but also non-intuitive, which may lead to easy confusion at the user side and limit its advertised functions of the multiple modes.

Therefore, what is desired is an intuitive and user friendly device with a mechanism enabling a transmission plate to provide vibrations in different modes which can be switched electronically. With this mechanism, a user can focuses more on the skin beautification process and its results, rather than operating the device itself.

### SUMMARY OF THE INVENTION

One object of this invention is to generate ultrasonic vibrations at multiple frequencies on a same ultrasound transmission plate.

Another object of this invention is to match the different ultrasonic transducer's frequency with a corresponding different plate thickness, such that the plate area where each transducer resides has a thickness that is matching to the ultrasound mode of that transducer and is most efficient for transmission of the ultrasonic vibration at the frequency of the transducer.

The present invention teaches an ultrasonic device with an ultrasonic vibration transmission plate being excited by multiple ultrasonic transducers. Multiple ultrasonic modes excitation on the same plate is realized by patterning the base plate thickness such that each different thickness of the plate matches to the frequency of the ultrasonic transducer residing on that thickness. This design provides feasibility of single plate excitation by multiple transducers and delivers easy usage and better skin care results.

In the preferred embodiment of the invention, the ultrasonic device includes: (1) at least one ultrasonic vibration transmission plate with at least one surface of the plate being in contact with a target surface; (2) at least two ultrasonic vibration generation transducers with each having a different frequency between 20 kHz to 25 MHz being in physical contact with the transmission plate; (3) different areas on the transmission plate where different ultrasonic transducers reside have different thicknesses for each transducer having a different frequency than the other ones.

In one implementation of the ultrasonic device according to the present invention, each different plate thickness under each ultrasonic transducer matches to the frequency of that transducer to transmit ultrasonic vibration more efficiently from the transducer to the skin.

In another implementation of the ultrasonic device according to the present invention, the different plate thickness can be realized by creating recession well into the plate where the transducer resides within the well and in contact with the bottom surface of the well.

In another implementation of the ultrasonic device according to the present invention, the different plate thickness can be realized by creating an extrusion on top of the plate where the transducer resides on top surface of the extrusion.

In another implementation of the ultrasonic device according to the present invention, the different transducers are arranged on the plate side by side. Further, the different transducers contain at least one transducer having a first frequency being neighbored by multiple transducers having a second frequency.

In another implementation of the ultrasonic device according to the present invention, the different transducers can contact the plate with a concentric arrangement, where at least one of the transducer, i.e. the first transducer, is a ring type with a center clearance, where at least one of the other transducers can reside within the ring type first transducer center clearance.

In another implementation of the ultrasonic device according to the present invention, the different transducers can contact the plate with at least one of the transducer, i.e. the first transducer, has a center clearance, where at least one of the other transducers resides within the first transducer center clearance, where the outside shape of the first transducer, the shape of the clearance and the shape of the other transducers can be any regular shape, for example ellipse, triangle, rectangle or square, or any other arbitrary irregular shape.

Yet in another implementation of the ultrasonic device according to the present invention, the different transducers can contact the plate with at least one of the transducer, i.e. the first transducer, has a clearance that makes the first transducer being a partially enclosed shape, where at least one of the other transducers resides within the first transducer clearance, where the outside shape of the first transducer, the shape of the clearance and the shape of the other transducers can be any arbitrary regular shape, for example ellipse, triangle, rectangle or square, or any other arbitrary irregular shape.

### BRIEF DESCRIPTION OF THE DRAWINGS

FIG. 1 is schematic diagram illustrating an ultrasonic transducer according to the prior art;

FIG. 2A is a schematic diagram illustrating a top view of the transducer according to the first preferred embodiment of the present invention;

FIG. 2B is a schematic diagram illustrating a cross-sectional view of the ultrasonic transducer of FIG. 2A;

FIG. 3A is a schematic diagram illustrating a top view of the transducer according to the second preferred embodiment of the present invention;

FIG. 3B is a schematic diagram illustrating a cross-sectional view of the ultrasonic transducer of FIG. 3A;

FIG. 4A is a schematic diagram illustrating a top view of the transducer according to the third preferred embodiment of the present invention; and

FIG. 4B is a schematic diagram illustrating a cross-sectional view of the ultrasonic transducer of FIG. 4A;

FIG. 5A is a schematic diagram illustrating a top view of the transducer according to the fourth preferred embodiment of the present invention;

FIG. 5B is a schematic diagram illustrating a cross-sectional view of the ultrasonic transducer of FIG. 5A;

FIG. 6A is a schematic diagram illustrating a top view of the transducer according to the fifth preferred embodiment of the present invention;

FIG. 6B is a schematic diagram illustrating a cross-sectional view of the ultrasonic transducer of FIG. 6A;

FIG. 7A is a schematic diagram illustrating a top view of the transducer according to the sixth preferred embodiment of the present invention;

FIG. 7B is a schematic diagram illustrating a cross-sectional view of the ultrasonic transducer of FIG. 7A;

FIG. 8A is a schematic diagram illustrating a top view of the transducer according to the seventh preferred embodiment of the present invention;

FIG. 8B is a schematic diagram illustrating a cross-sectional view of the ultrasonic transducer of FIG. 8A;

FIG. 9A is a schematic diagram illustrating a top view of the transducer according to the eighth preferred embodiment of the present invention; and

FIG. 9B is a schematic diagram illustrating a cross-sectional view of the ultrasonic transducer of FIG. 9A.

FIG. 10A is a schematic diagram illustrating a top view of the transducer according to the ninth preferred embodiment of the present invention;

FIG. 10B is a schematic diagram illustrating a cross-sectional view of the ultrasonic transducer of FIG. 10A;

FIG. 11A is a schematic diagram illustrating a top view of the transducer according to the tenth preferred embodiment of the present invention; and

FIG. 11B is a schematic diagram illustrating a cross-sectional view of the ultrasonic transducer of FIG. 11A.

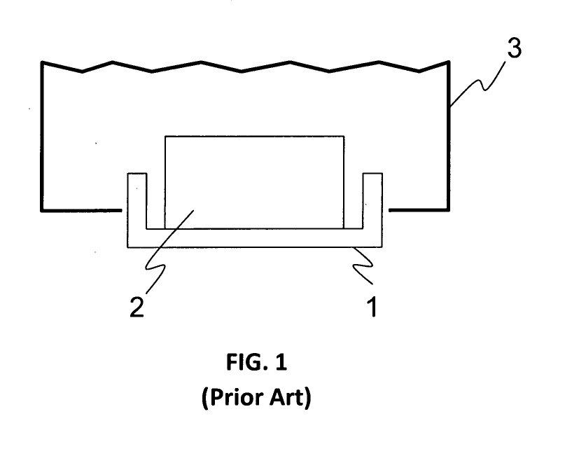

### DESCRIPTION OF THE INVENTION

While the present invention may be embodied in many different forms, designs or configurations, for the purpose of promoting an understanding of the principles of the invention, reference will be made to the embodiments illustrated in the drawings and specific language will be used to describe the same. It will nevertheless be understood that no limitation of the scope of the invention is thereby intended. Any alterations and further implementations of the principles of the invention as described herein are contemplated as would normally occur to one skilled in the art to which the invention relates.

The present invention teaches an ultrasonic device for skin care massage, comprising one or more ultrasonic vibration transmission plates, each of which having a backside and a smooth surface for contacting human skin; and at least two different ultrasonic vibration transducers coupled to a backside of each of the ultrasonic vibration plates. Each of the ultrasonic vibration transducers generates an ultrasonic wave with a unique frequency between 20 kHz to 25 MHz. Each of the ultrasonic vibration transducers has a bottom surface paralleling to the smooth surface. A distance between the bottom surface of any of the ultrasonic vibration transducers to the smooth surface is approximately an integer times of the half wavelength of the ultrasonic wave generated by the ultrasonic vibration transducer. In the context of this application, a plate thickness matching with the transducer's frequency means that when an ultrasonic transducer is positioned on a vibration transmission plate surface, the thickness of the vibration transmission plate under the transducer matches with the frequency of the ultrasonic transducer for maximally transmitting the vibrations generated by the transducer. In other words, at the matching thickness, the vibration transmission plate can most efficiently transmit the ultrasonic vibration generated by the transducer across the thickness of the plate to the other side of the plate opposing the side where the transducer resides. Such thickness is usually N times of the half wavelength of the intrinsic sound wave of the plate at the frequency where the ultrasonic transducer operates, where N is a non-zero integer.

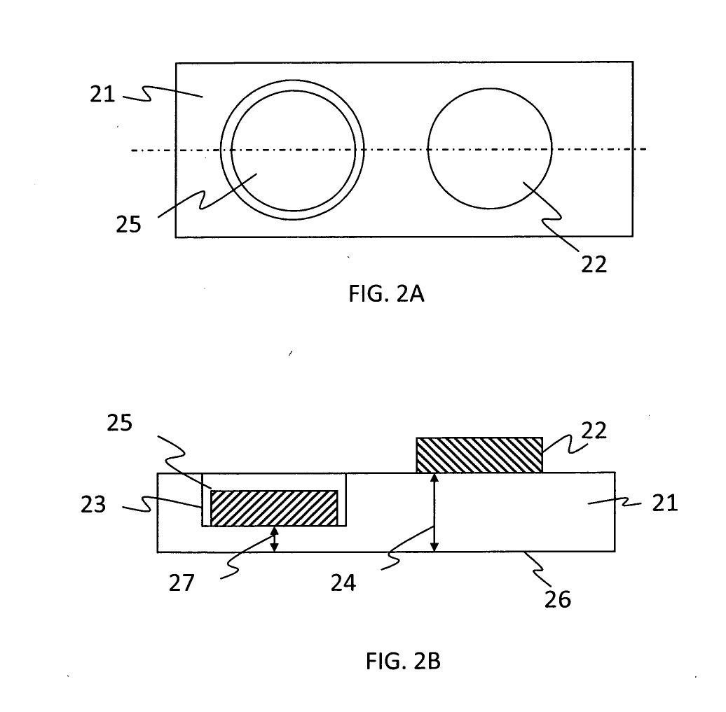

FIG. 2A is a schematic diagram illustrating a top view of the ultrasonic transmission device, called transducer, according to the first preferred embodiment of the present invention, and FIG. 2B is a schematic diagram illustrating a cross-sectional view of the transducer of FIG. 2A. The ultrasonic transducer includes (1) a plate 21; (2) a lower frequency transducer (TLF) 22; (3) a first plate thickness 24 between the bottom surface of the TLF 22 and the treatment surface 26 of the plate 21; (4) a higher frequency transducer (THF) 25 residing in a counterbore 23 in the plate 21; and (5) a second plate thickness 27 between the bottom surface of the THF 25 and the treatment surface 26 of the plate 21.

TLF 22 and THF 25 are incorporated in the same plate side by side with the plate base thickness matching TLF frequency, while THF 25 resides within a counterbore type well structure 23 recessed into the plate 21. The first plate thickness 24 of the plate area where THF 25 resides matches with the THF frequency. In implementations, there can be multiple THFs neighboring a single TLF or multiple TLFs neighboring a single THF. Both THF25 and TLF 22 are in a shape of circular column.

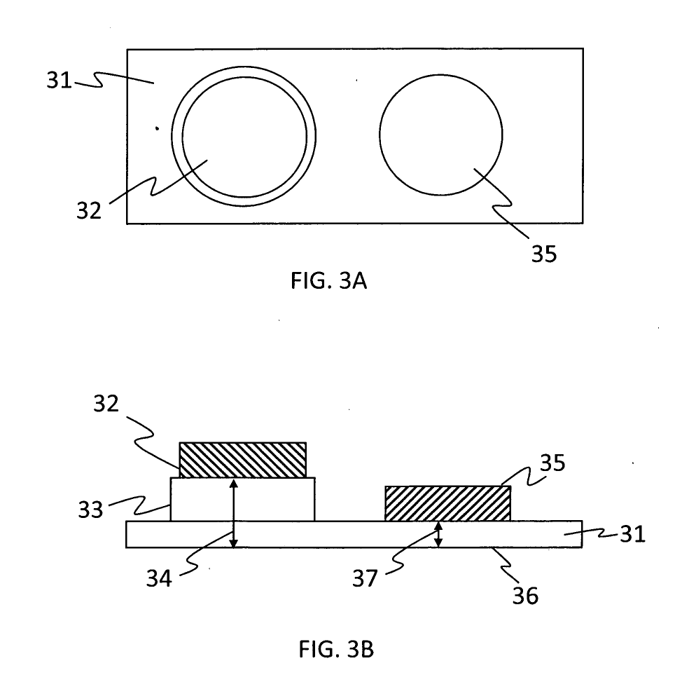

FIG. 3A is a schematic diagram illustrating a top view of the ultrasonic transmission device, called transducer, according to the second preferred embodiment of the present invention, and FIG. 3B is a schematic diagram illustrating a side cross-sectional view of the transducer of FIG. 3A. The ultrasonic transducer includes (1) a plate 31; (2) a lower frequency transducer (TLF) 32; (3) a first plate thickness 34 between the bottom surface of the TLF 32 and the treatment surface 36 of the plate 31; (4) a higher frequency transducer (THF) 35; and (5) a second plate thickness 37 between the bottom surface of the THF 35 and the treatment surface 36 of the plate 31.

TLF 32 and THF 35 are incorporated in the same plate side by side with the plate base thickness matching with THF frequency, while TLF 32 sits on an extrusion 33 arising from the base plate 31. The extrusion 33 and base plate 31 form an effective thickness 34 that matches with the TLF frequency. In implementations, there can be multiple THFs neighboring a single TLF or multiple TLFs neighboring a single THF. Both THF35 and TLF 32 are in a shape of circular column.

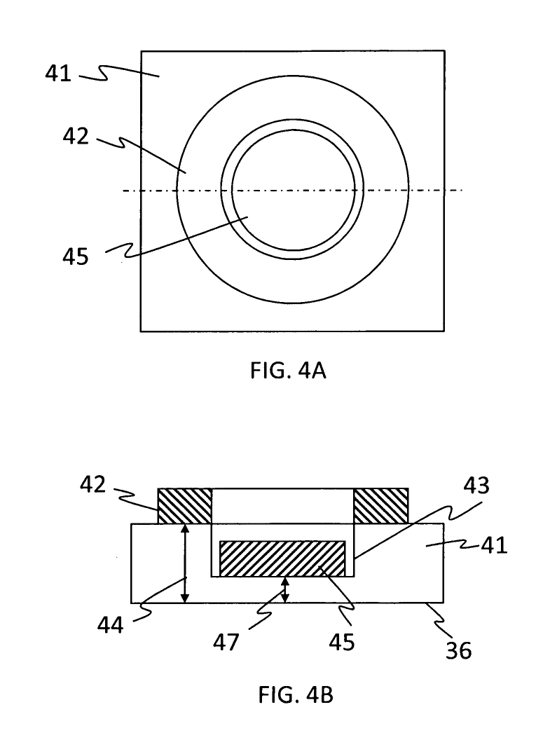

FIG. 4A is a schematic diagram illustrating a top view of the ultrasonic transmission device, called transducer, according to the third preferred embodiment of the present invention, and FIG. 4B is a schematic diagram illustrating a side cross-sectional view of the transducer of FIG. 4A. The ultrasonic transducer includes (1) a plate 41; (2) a lower frequency transducer (TLF) 42; (3) a first plate thickness 44 between the bottom surface of the TLF 42 and the treatment surface 46 of the plate 41; (4) a higher frequency transducer (THF) 45; and (5) a second plate thickness 47 between the bottom surface of the THF 45 and the treatment surface 46 of the plate 41.

TLF 42 and THF 45 incorporated in the same plate 41 with a concentric annular arrangement, where TLF 42 is a ring type transducer and THF 45 is a smaller round transducer that resides within a counterbore well 43 recessed into the base plate 41 located within the center clearance of the TLF 42. The first plate thickness 44, i.e. the plate base thickness, matches with the TLF frequency. The second plate thickness 47, i.e. the effective plate thickness, matches with the THF frequency. Both THF45 and TLF 42 are in a shape of circular column.

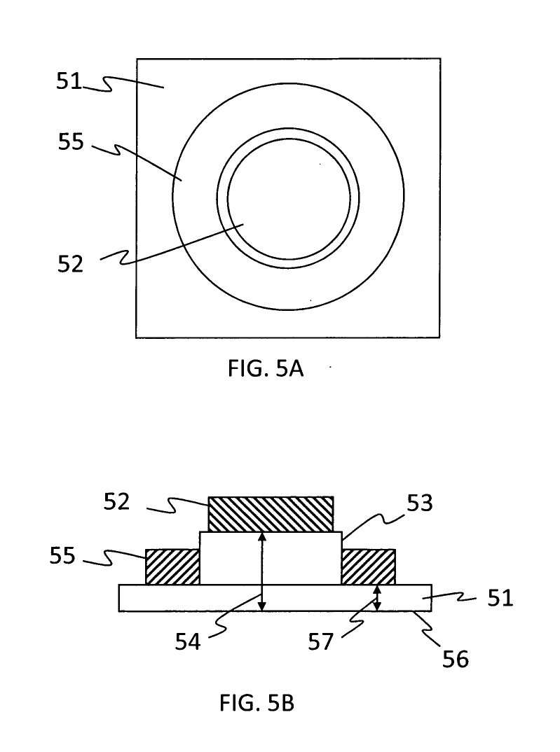

FIG. 5A is a schematic diagram illustrating a top view of the ultrasonic transmission device, called transducer, according to the fourth preferred embodiment of the present invention, and FIG. 5B is a schematic diagram illustrating a side cross-sectional view of the transducer of FIG. 5A. The ultrasonic transducer includes (1) a plate 51; (2) a lower frequency transducer (TLF) 52; (3) a first plate thickness 54 between the bottom surface of the TLF 52 and the treatment surface 56 of the plate 51; (4) a higher frequency transducer (THF) 55; and (5) a second plate thickness 57 between the bottom surface of the THF 55 and the treatment surface 56 of the plate 51.

TLF 52 and THF 55 are incorporated in the same plate 51 with a concentric arrangement, where THF 55 is a ring type transducer and TLF 52 is a smaller transducer that sits on top of an extrusion 53 arising from the base plate 51. The extrusion 53 is located within the center clearance of the THF 55. The second plate thickness 57, i.e. the plate base thickness, matches with the THF frequency. The first plate thickness 54, i.e. the effective plate thickness, matches with the TLF frequency. Both THF55 and TLF 52 are in a shape of circular column.

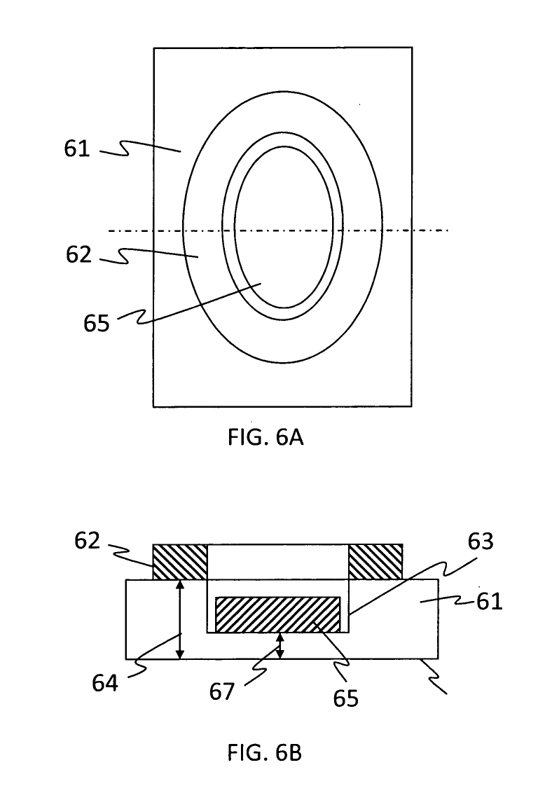

FIG. 6A is a schematic diagram illustrating a top view of the ultrasonic transmission device, called transducer, according to the fifth preferred embodiment of the present invention, and FIG. 6B is a schematic diagram illustrating a side cross-sectional view of the transducer of FIG. 6A. The ultrasonic transducer includes (1) a plate 61; (2) a lower frequency transducer (TLF) 62; (3) a first plate thickness 64 between the bottom surface of the TLF 62 and the treatment surface 66 of the plate 61; (4) a higher frequency transducer (THF) 65; and (5) a second plate thickness 67 between the bottom surface of the THF 65 and the treatment surface 66 of the plate 61.

TLF 62 and THF 65 are incorporated in the same plate 61 with TLF 62 having a center clearance and THF 65 is a smaller transducer that resides within a counterbore well 63 recessed into the base plate 61 located within the center clearance of the TLF 62. The first plate thickness 64, i.e. the plate base thickness, matches with the TLF frequency. The second plate thickness 67, i.e. the effective plate thickness, matches with THF frequency. THF65 is in a shape of column with a top view of ellipse. TLF62 is in a shape of elliptical cylinder surrounding THF65.

Although in FIG. 6A, TLF62, the center clearance and THF 65 are shown in elliptical shapes, they can be any regular and irregular shape in practice.

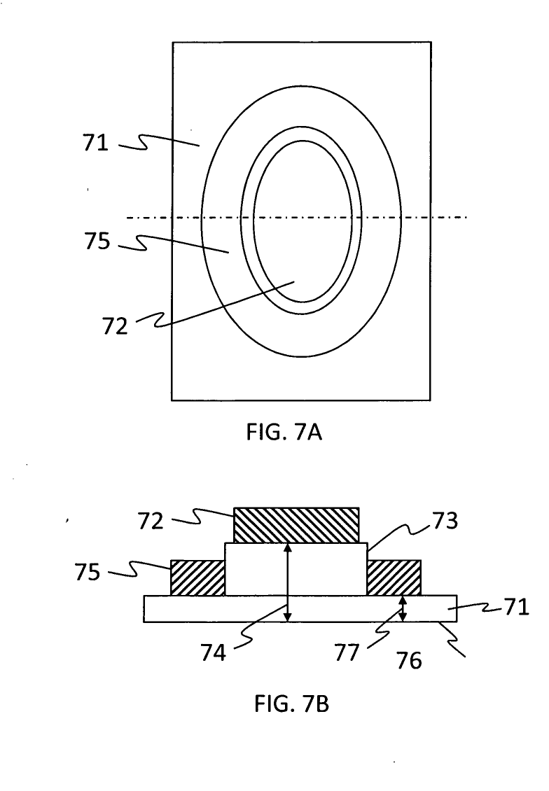

FIG. 7A is a schematic diagram illustrating a top view of the ultrasonic transmission device, called transducer, according to the sixth preferred embodiment of the present invention, and FIG. 7B is a schematic diagram illustrating a side cross-sectional view of the transducer of FIG. 7A. The ultrasonic transducer includes (1) a plate 71; (2) a lower frequency transducer (TLF) 72; (3) a first plate thickness 74 between the bottom surface of the TLF 72 and the treatment surface 76 of the plate 71; (4) a higher frequency transducer (THF) 75; and (5) a second plate thickness 77 between the bottom surface of the THF 75 and the treatment surface 76 of the plate 71.

TLF 72 and THF 75 are incorporated in the same plate 71. THF 75 has a center clearance and TLF 72 is a smaller transducer that sits on top of an extrusion 73 arising from the base plate 71. The extrusion 73 is located within the center clearance of the THF 75. The second plate thickness 77, i.e. the plate base thickness, matches with the THF frequency. The first plate thickness 74, i.e. the effective plate thickness ZLF, matches with the TLF frequency. TLF72 is in a shape of column with a top view of ellipse. THF75 is in a shape of elliptical cylinder surrounding TLF72.

Although in FIG. 7A, TLF 72, the center clearance and THF 75 are shown in shapes of ellipse, they can be any regular and irregular shape in practice.

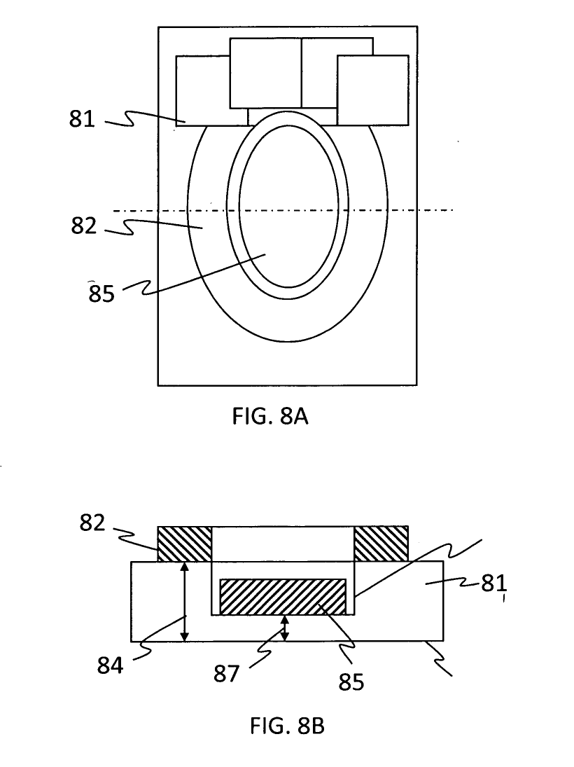

FIG. 8A is a schematic diagram illustrating a top view of the ultrasonic transmission device, called transducer, according to the seventh preferred embodiment of the present invention, and FIG. 8B is a schematic diagram illustrating a side cross-sectional view of the transducer of FIG. 8A. The ultrasonic transducer includes (1) a plate 81; (2) a lower frequency transducer (TLF) 82; (3) a first plate thickness 84 between the bottom surface of the TLF 82 and the treatment surface 86 of the plate 81; (4) a higher frequency transducer (THF) 85; and (5) a second plate thickness 87 between the bottom surface of the THF 85 and the treatment surface 86 of the plate 81.

TLF 82 and THF 85 are incorporated in the same plate 81. TLF 82 has a clearance that makes TLF 82 a partially enclosed shape. THF 85 is a smaller transducer that resides within a counterbore well 83 recessed into the base plate 81 located within the clearance of the TLF 82. The first plate thickness 84, i.e. the plate base's larger thickness, matches with the TLF frequency. The second plate thickness 87, i.e. the effective plate thickness 87 matches with the THF frequency. THF85 is in a shape of column with a top view of ellipse. TLF82 is in a shape of partial elliptical cylinder substantially surrounding THF85.

Although in FIG. 8A, TLF 82, the center clearance and THF 85 are shown in shapes of ellipse or partial ellipse, they can be any regular and irregular shape in practice.

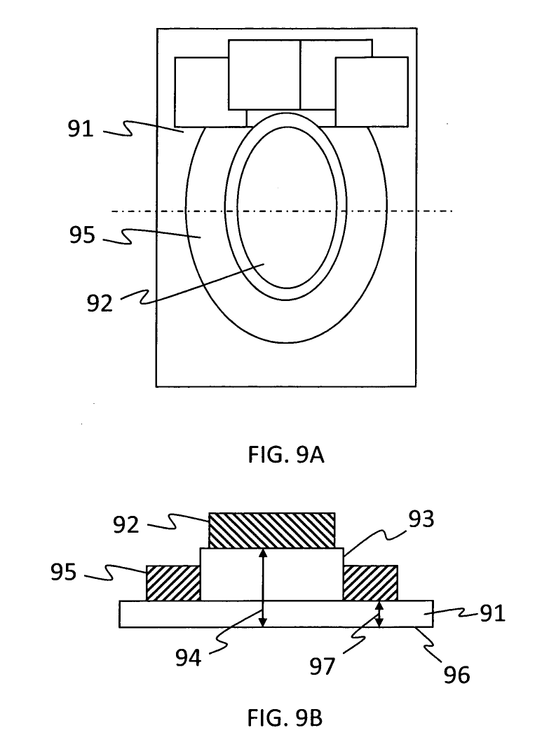

FIG. 9A is a schematic diagram illustrating a top view of the ultrasonic transmission device, called transducer, according to the eighth preferred embodiment of the present invention, and FIG. 9B is a schematic diagram illustrating a side cross-sectional view of the transducer of FIG. 9A. The ultrasonic transducer includes (1) a plate 91; (2) a lower frequency transducer (TLF) 92; (3) a first plate thickness 94 between the bottom surface of the TLF 92 and the treatment surface 96 of the plate 91; (4) a higher frequency transducer (THF) 95; and (5) a second plate thickness 97, i.e. the plate's smaller thickness, between the bottom surface of the THF 95 and the treatment surface 96 of the plate 91.

TLF 92 and THF 95 are incorporated in the same plate 91. THF 95 has a clearance that makes THF 95 a partially enclosed shape. TLF 92 is a smaller transducer that sits on top of an extrusion 93 arising from the base plate 91. The extrusion 93 is located within the center clearance of the THF 95. The second plate thickness 97, i.e. the plate's base thickness, matches with the THF frequency. The first plate thickness 94, i.e. the effective plate thickness, matches with the TLF frequency. TLF 92 is in a shape of column with a top view of ellipse. THF 95 is in a shape of partial elliptical cylinder substantially surrounding TLF 92.

Although in FIG. 9A, the TLF 92, the center clearance and the THF 95 are shown in shapes of ellipse or partial ellipse, they can be any regular and irregular shape in practice.

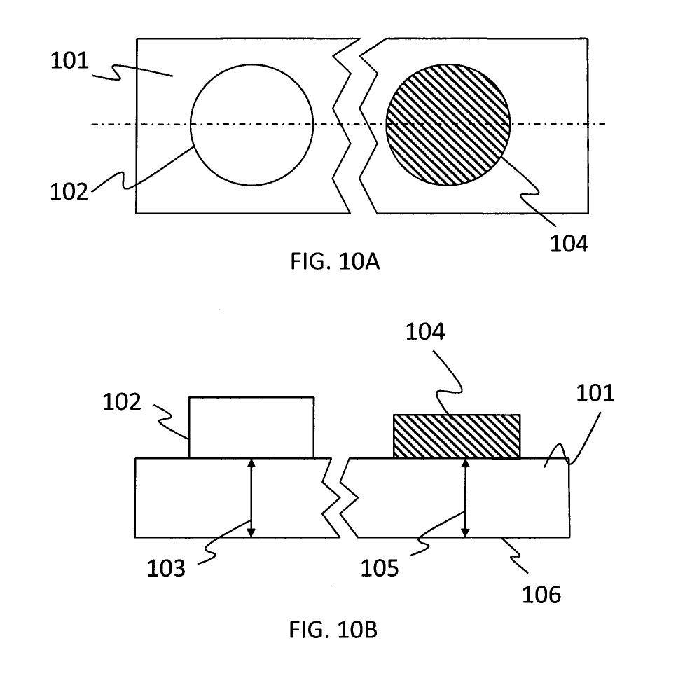

FIG. 10A is a schematic diagram illustrating a top view of the ultrasonic transmission device, called transducer, according to the ninth preferred embodiment of the present invention, and FIG. 10B is a schematic diagram illustrating a cross-sectional view of the transducer of FIG. 10A. The ultrasonic transducer includes (1) a plate 101; (2) a transducer with a first frequency (TF1) 102; (3) a first plate thickness 103 between the bottom surface of the TF1 102 and the treatment surface 106 of the plate 101; (4) a transducer with a second frequency (TF2) 104, that is different from the first frequency; and (5) a second plate thickness 105 between the bottom surface of the TF2 104 and the treatment surface 106 of the plate 101.

TF1 102 and TF2 104 are incorporated on the same plate side. The first plate thickness 103 matches with the ultrasonic frequency of TF1 102. The second plate thickness 105 matches with the ultrasonic frequency of TF2 104. In implementations, there can be multiple TF1s 102 neighboring a single TF2 104 or multiple TF2s 104 neighboring a single TF1 102. Alternatively, a plurality of TF1s 102 and TF2s 104 can be arranged in an intermixed matrix. A plurality of TF1s 102 and TF2s 104 can also be arranged in separate clusters with each cluster having same frequency transducers, and different clusters are positioned on different areas on the same plate. In all possible arrangements, each transducer resides on a plate thickness that matches to the transducers ultrasonic frequency.

Different than in FIGS. 2A and 2B, where high frequency transducer sits on a plate thickness thinner than the low frequency transducer, with the plate thickness under each transducer being an integer times of half wavelength at the frequency of each transducer, high frequency transducer in FIGS. 10A and 10B may sit on a thicker plate thickness than the low frequency transducer. One such example can be the plate thickness under the high frequency transducer being full sound wavelength at the high frequency, while the plate thickness under the low frequency transducer being half sound wavelength at the low frequency. With the low frequency value larger than 50% of the high frequency value, the plate thickness under the low frequency transducer is thinner than the plate thickness under the high frequency transducer.

Although in FIG. 10A, the TF1 and TF2 are shown as circular shape, the actual shapes of TF1 and TF2 can be any regular or irregular shapes.

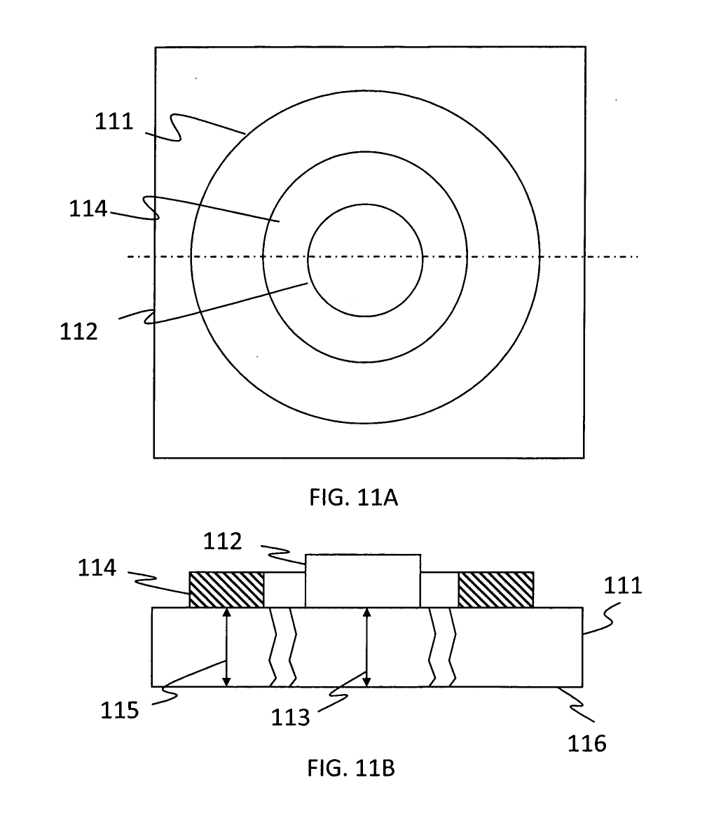

FIG. 11A is a schematic diagram illustrating a top view of the ultrasonic transmission device, called transducer, according to the tenth preferred embodiment of the present invention, and FIG. 11B is a schematic diagram illustrating a cross-sectional view of the transducer of FIG. 11A. The ultrasonic transducer includes (1) a plate 111; (2) a transducer with a first frequency (TF1) 112; (3) a first plate thickness 113 between the bottom surface of the TF1 112 and the treatment surface 116 of the plate 111; (4) a transducer with a second frequency (TF2) 114 that is different from the first frequency; and (5) a second plate thickness 115 between the bottom surface of the TF2 114 and the treatment surface 116 of the plate 111.

TF1 112 and TF2 114 are incorporated on the same plate side. TF2 114 has a center clearance. TF1 112 is a smaller transducer that resides in the center clearance of the TF2 114. The first plate thickness 113 matches with the ultrasonic frequency of TF1 112. The second plate thickness 115 matches with the ultrasonic frequency of TF2 114. TF1 112 is in a shape of circular column. TF2 114 is in a shape of annular cylinder surrounding TF1 112.

Different than in FIGS. 3A and 3B, where high frequency transducer sits on a plate thickness thinner than the low frequency transducer, with the plate thickness under each transducer being an integer times of half wavelength at the frequency of each transducer, high frequency transducer in FIGS. 10A and 10B may sit on a thicker plate thickness than the low frequency transducer. One such example can be the plate thickness under the high frequency transducer being full sound wavelength at the high frequency, while the plate thickness under the low frequency transducer being half sound wavelength at the low frequency. With the low frequency value larger than 50% of the high frequency value, the plate thickness under the low frequency transducer is thinner than the plate thickness under the high frequency transducer.

Although in FIG. 11A, the TF2 and its center clearance are shown as circular shape, the actual shapes of TF1, TF2 and the center clearance of TF2 can be any regular and irregular shape in practice. There can also be multiple TF1 transducers residing in a single TF2 transducer center clearance. In all possible arrangements, each transducer resides on a plate thickness that matches with the transducers ultrasonic frequency.

In the above described embodiments, each of the ultrasonic vibration transducers generates an ultrasonic wave with a unique frequency between 20 kHz to 25 MHz. Ideally, the distance between the bottom surface of an ultrasonic vibration transducer to the smooth treatment surface is an integer times of the half wavelength of the ultrasonic wave generated by the ultrasonic vibration transducer. In implementation, each plate thickness under each ultrasonic transducer may be within −20% and +20% variation of an integer times of the half wavelength of the sound wave traveling within the ultrasonic vibration plate at a frequency same as the frequency of the wave generated by the ultrasonic transducer.

While one or more embodiments of the present invention have been illustrated above, the skilled artisan will appreciate that modifications and adoptions to those embodiments may be made without departing from the scope and spirit of the present invention.

1. An ultrasonic device for skin care massage, comprising:
one or more ultrasonic vibration transmission plates, each of which having a backside and a smooth surface for contacting human skin; and
at least two different ultrasonic vibration transducers coupled to a backside of each of said ultrasonic vibration plates;
wherein each of said ultrasonic vibration transducers generates an ultrasonic wave with a unique frequency between 20 kHz to 25 MHz;
wherein each of said ultrasonic vibration transducers has a bottom surface paralleling to said smooth surface;
wherein a distance between said bottom surface of any of said ultrasonic vibration transducers to said smooth surface is approximately an integer times of half wavelength of said ultrasonic wave generated within said vibration plates by said ultrasonic vibration transducer.

2. The device according to claim 1, wherein said ultrasonic vibration transducers comprise at least one first ultrasonic vibration transducer which is embedded in said ultrasonic vibration plate and at least one second ultrasonic vibration transducer which is coupled to said ultrasonic vibration plate's back surface.

3. The device according to claim 2, wherein said first ultrasonic vibration transducer is in a shape of circular column and said second ultrasonic vibration transducer is in a shape of annular cylinder surrounding said first ultrasonic vibration transducer.

4. The device according to claim 2, wherein said first ultrasonic vibration transducer is in a shape of elliptical column and said second ultrasonic vibration transducer is in a shape of elliptical cylinder surrounding said first ultrasonic vibration transducer.

5. The device according to claim 2, wherein said first ultrasonic vibration transducer is in a shape of elliptical column and said second ultrasonic vibration transducer is in a shape of partial elliptical cylinder substantially surrounding said first ultrasonic vibration transducer.

6. The device according to claim 2, wherein said first ultrasonic vibration transducer is in a shape of column with any type of top view and said second ultrasonic vibration transducer is in a shape of a cylinder with any top view, said second ultrasonic vibration transducer surrounding said first ultrasonic vibration transducer.

7. The device according to claim 1, wherein said ultrasonic vibration transducers comprise at least one first ultrasonic vibration transducer which is coupled to an extruding member of said ultrasonic vibration plate and at least one second ultrasonic vibration transducer which is coupled to said ultrasonic vibration plate's back surface.

8. The device according to claim 7, wherein said first ultrasonic vibration transducer is in a shape of circular column and said second ultrasonic vibration transducer is in a shape of annular cylinder surrounding said first ultrasonic vibration transducer.

9. The device according to claim 7, wherein said first ultrasonic vibration transducer is in a shape of elliptical column and said second ultrasonic vibration transducer is in a shape of elliptical cylinder surrounding said first ultrasonic vibration transducer.

10. The device according to claim 7, wherein said first ultrasonic vibration transducer is in a shape of elliptical column and said second ultrasonic vibration transducer is in a shape of partial elliptical cylinder substantially surrounding said first ultrasonic vibration transducer.

11. The device according to claim 7, wherein said first ultrasonic vibration transducer is in a shape of column with any type of top view and said second ultrasonic vibration transducer is in a shape of a cylinder with any top view, said second ultrasonic vibration transducer surrounding said first ultrasonic vibration transducer.

12. The device according to claim 1, wherein said different ultrasonic vibration transducers are arranged side by side on each of said vibration transmission plates.

13. The device according to claim 1, wherein said different ultrasonic vibration transducers are arranged in intermixed matrix on each of said vibration transmission plates.

14. The device according to claim 1, wherein said different ultrasonic vibration transducers are arranged in various clusters on each of said vibration transmission plates.

15. The device according to claim 14, wherein said ultrasonic vibration transducers in each cluster have a same ultrasonic frequency.
